(19) United States
(12) Patent Application Publication
(10) Pub. No.: US 2026/0370954 A1
(43) Pub. Date: Nov. 19, 2026

(54) METHOD FOR EVALUATING THE SUITABILITY OF AUTO-EXPOSURE SETTINGS OF A MOBILE DEVICE FOR PERFORMING A COLOR BASED MEASUREMENT

(71) Applicant: Roche Diabetes Care, Inc., Indianapolis, IN (US)

(72) Inventors: Max Berg, Mannheim (DE); Fredrik Hailer, Mannheim (DE); Bernhard Limburg, Mannheim (DE)

(21) Appl. No.: 19/721,066

(22) Filed: Jun. 26, 2026

Related U.S. Application Data
(63) Continuation of application No. PCT/EP2024/087555, filed on Dec. 19, 2024.

(30) Foreign Application Priority Data
Dec. 31, 2023 (EP) 23220879.3

(51) Int. Cl. H04N 23/73; G01J 3/02; G01J 3/50; H04N 23/61; H04N 23/71

(52) U.S. Cl. CPC H04N 23/73; G01J 3/027; G01J 3/50; H04N 23/61; H04N 23/71

(57) ABSTRACT

A method of evaluating the suitability of auto-exposure settings of a mobile device for performing a color based measurement, the mobile device having at least one camera for capturing an image representative of a scene.

### CROSS REFERENCE TO RELATED APPLICATIONS

This is a continuation of application serial no. PCT/EP 2024/087555 filed Dec. 19, 2024, which claims priority to EP 23 220 879.3 filed Dec. 31, 2023, the entire disclosures of each of which are hereby incorporated herein by reference.

### BACKGROUND

### Technical Field

The present disclosure refers to a method for evaluating the suitability of auto-exposure settings of a mobile device for performing a color based measurement. The disclosure further relates to a mobile device having at least one camera, and to a computer program. The methods, devices, and computer programs specifically may be used in medical diagnostics, in order to for example qualitatively and/or quantitatively detect one or more analytes in one or more body fluids, such as for detecting glucose in blood or another (bodily) fluid. Other fields of application of the present disclosure, however, are also feasible.

In the field of medical diagnostics, in many cases, one or more analytes have to be detected in samples of a body fluid, such as blood, interstitial fluid, urine, saliva or other types of body fluids. Examples of analytes to be detected are glucose, triglycerides, lactate, ketone, cholesterol or other types of analytes. According to the concentration and/or the presence of the analyte, an appropriate treatment may be chosen, if necessary. Without narrowing the scope, the disclosure specifically may be described with respect to blood glucose measurements. It shall be noted, however, that the present disclosure may also be used for other types of analytical measurements.

### Background Art

Generally, devices and methods known to the skilled person make use of test strips comprising one or more test chemistries, which, in presence of the analyte to be detected, are capable of performing one or more detectable detection reactions, such as optically detectable detection reactions. With regard to these test chemistries, reference may be made e.g. to J. Hoenes et al.: The Technology Behind Glucose Meters: Test Strips, Diabetes Technology & Therapeutics, Volume 10, Supplement 1, 2008, S-10 to S-26. Other types of test chemistry are possible and may be used for performing the present disclosure.

Typically, one or more optically detectable changes in the test chemistry are monitored, in order to derive the concentration of the at least one analyte to be detected from these changes. For detecting the at least one change of optical properties of the test field of a test strip, various types of detectors (with various types of light sources for illuminating the test fields) are known (also referred to as dedicated meters or analytical measurement devices).

Further, besides using customized detectors which are specifically developed for the purpose of optically detecting changes in the test chemistry comprised by corresponding test strips, recent developments aim at using widely available devices such as smartphones. However, when consumer-electronics devices having a camera, such as smartphones, are employed instead of dedicated analytical measurement devices in order to determine analyte concentrations various influences need to be taken into account. As an example, lighting conditions, positioning, or other more or less uncontrollable conditions or settings are to be considered.

US20170042451A1 describes that pixel values in an image taken with a computing device which can be a smartphone are heavily dependent on the lighting that existed when the image was taken, and on the exposure of the image taken by the camera. It is therefore suggested to take an image with a reference card also visible in the image. A location on the card having a pure gray color of a known reflectivity is then identified in the image that was taken and pixels on this card location are analyzed in order to standardize both the exposure and the white balance of the image. The standardization is then applied to an average value of selected pixels of the image, in order to create a corrected pixel value.

U.S. Pat. No. 9,903,857B2 describes a testing apparatus for performing an assay, the testing apparatus comprising: a receptacle containing a reagent, the reagent being reactive to an applied test sample by developing a color or pattern variation; a mobile phone or a laptop, comprising a processor and an image capture device, wherein the processor is configured to process data captured by the image capture device and output a test result for the applied test sample. It is further described that where the dynamic range of the resulting image is inadequate for the limit of detection, or where the constraints on lighting mean exposure settings force the dynamic range to be too poor it may be desirable to capture multiple images at different exposure settings and then combine these into a single higher dynamic range ‘virtual’ image, using appropriate algorithms to reorient the images between frames, and to discard unwanted or low value data.

U.S. Pat. No. 9,506,855B2 discloses that a challenge for everyday use outside of a controlled setting for mobile phones for quantitative colorimetric analysis is compensating for ambient light conditions. It is described that when measuring 12 regions on a reference chart, it was found that the measured intensities between different ambient light conditions had a linear relationship. It is concluded that the reference color chart can be used for compensating the intensity difference caused by ambient light changes. It is also described that the reference chart nearly eliminates effects of automatic camera functions, making images reproducible and quantifiable.

WO2022/078977 refers to a method of controlling auto-exposure settings of a mobile device having at least one camera, and to a method of determining the concentration of an analyte in a sample by using the mobile device. The method comprises setting an exposure metering area and determining auto-exposure settings based on the scene in the set exposure metering area wherein the scene in the set exposure metering area comprises at least part of the reagent test field of the optical test strip having the sample applied thereto, and at least part of the plurality of different color reference fields and at least part of the one or more gray background fields of a color reference card, and capturing an image comprising the scene, wherein the determined auto-exposure settings are used.

### SUMMARY

Despite the advantages involved in using a mobile computing device for the purpose of performing an analytical measurement, several technical challenges remain. Specifically, the use of mobile devices to determine analyte concentrations using optical test strips may require an accurate determination of the color change of the optical test strip and, thus, often remains challenging. Appropriate image brightness needs to be ensured. The observed brightness in the captured images may, e.g., be dependent on various influencing factors, such as settings determining the exposure when capturing the image, such as the shutter speed, the aperture size, and sensor gain, as well as post-processing steps applied to the image after it was captured. Specifically, the determination of the exposure settings by the mobile devices is often a device-specific process conducted in an automatic exposure (auto-exposure) mode and, thus, may be unknown to the user or manufacturer of an application for performing a color based measurement with a mobile device.

Although an image taken with auto-exposure settings normally has a good overall or average brightness, there may be local under-or overexposures of image portions being much darker or much lighter than the image average. Such intensity values falling outside a minimum or maximum intensity will appear as uniform area of the minimum or maximum brightness in the image so that image details are lost. If essential image elements (e.g., the test field) are thus within those under-or overexposed image areas, this image cannot be used for determining an analyte concentration.

Camera systems, such as those of a smartphone, may provide options for the user or an app to be downloaded to the smartphone to set the exposure metering area to cover a sub-scene of a scene in the field of view of the camera so that the camera system should determine auto-exposure settings based on the sub-scene in the set exposure metering area only. However, smartphones with camera systems exist which let a user or the app set an exposure metering area (or rather pretend to the user or the app that the exposure metering area may be set to be used for determining auto-exposure settings only based thereon and not based on other parts of the scene in the field of view), but the actually used auto-exposure settings when an image is taken are nevertheless influenced by parts of the scene outside of the sub-scene to which the exposure metering area was set by the user or the app. In this case, the actually used exposure metering area for determining auto-exposure settings of the device thus is different from the exposure metering area set by the user or app. This is disadvantageous when an image of a test strip for performing an analytical measurement shall be captured with the smartphone and analyzed for determining the analyte concentration as it may lead to uncontrollable local under-or overexposures of relevant image portions, in particular when, specifically, the part of the image corresponding to the set metering area comprising the test strip shall be analyzed. Such intensity values falling outside a minimum or maximum intensity which can be represented will appear as uniform area of the minimum or maximum brightness in the image so that image details are lost hindering an accurate analytical measurement.

Where the user or app can influence the auto-exposure settings of a camera system of a smartphone in an expected way by setting a metering area, it is also likely that, even in case of an image having been taken that shows local under or over-exposure, better lighting conditions may provide for a suitable (non-over or non-under exposed) image. A respective recommendation can be given to the user of the smartphone. However, where the user's or app's influence on the auto-exposure settings by setting a metering area is unknown/uncontrolled, recommendations for capturing a more suitable image for performing a color based measurement with the smartphone are difficult to make.

As smartphones or other mobile device and their operating systems and medical applications for smartphones often times are developed by different entities this causes issue as described above as the app manufacturer may not be able to entirely control the camera system of the smartphone. Consequently, there exists a need of being able to determine whether the mobile device is suitable for performing a color based measurement with the mobile device, in particular whether the auto-exposure settings are suitable for performing a color based measurement.

It is therefore desirable to provide methods and devices which address the above mentioned technical challenges using mobile devices such as consumer-electronics mobile devices, specifically multipurpose mobile devices which are not dedicated to analytical measurements such as smartphones or tablet computers. Specifically, methods and devices shall be proposed for evaluating the suitability of auto-exposure settings of a mobile device to ensure acquisition of images suitable for reliable and accurate determination of an analyte concentration in a sample. It is further desirable to provide a method and devices which allow for a user-friendly suitability test of the mobile device for a mobile-based determination of an analyte in a sample of bodily fluid with high accuracy and reliability of the analytical measurement.

This problem is addressed by a method for evaluating the suitability of auto-exposure settings of a mobile device for performing a color based measurement, the mobile device having at least one camera for capturing an image representative of a scene, a computer program, and a mobile device with features described herein. Advantageous embodiments which might be realized in an isolated fashion or in any arbitrary combinations are also disclosed herein.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect, a method, preferably a computer-implemented method, for evaluating the suitability of auto-exposure settings of a mobile device for performing a color based measurement, the mobile device having at least one camera for capturing an image representative of a scene, is disclosed.

The method, in one embodiment, comprises the following steps:
a1) receiving first exposure metering area settings to cover a first sub-scene of a first scene in the field of view of the camera;
a2) capturing a first image of the first scene;
a3) determining a first intensity parameter from the first image;
b1) receiving second exposure metering area settings to cover a second sub-scene of a second scene or the entire second scene in the field of view of the camera;
b2) capturing a second image of the second scene;
b3) determining a second intensity parameter from the second image;
c) determining a suitability parameter by using the first intensity parameter and the second intensity parameter,
wherein the suitability parameter being outside a predefined suitability range indicates non-suitability of the auto-exposure settings of the mobile device for performing a color based measurement and initiates a non-suitability action; and/or
wherein the suitability parameter being inside a predefined suitability range indicates suitability of the auto-exposure setting of the mobile device for performing a color based measurement and initiates a suitability action,
wherein either the second exposure metering area settings are different from the first exposure metering area settings, or the second scene is different from the first scene.

The method may comprise further steps which are not listed.

The term “color based measurement” as used herein is a broad term and is to be given its ordinary and customary meaning to a person of ordinary skill in the art and is not to be limited to a special or customized meaning. The term may specifically refer to an analyte measurement based on a color formation reaction, wherein the color formation reaction occurs in a test field of a test strip after a sample has been applied to the test field of the test strip. The term color formation reaction may specifically refer to a chemical, biological or physical reaction during which a color, specifically a reflectance, of at least one element involved in the reaction, changes with the progress of the reaction. Thus, as an example, reference may be made to above mentioned biochemical reactions which typically are used for detecting blood glucose, involving a color change. Other types of color changing or color formation reactions are known to the skilled person, such as typical chemical reactions for determining the pH value. The color based measurement may in particular comprise determining an analyte concentration, such as determining an analyte concentration in a sample applied to a test field of a test strip.

The term “auto-exposure settings” as used herein is a broad term and is to be given its ordinary and customary meaning to a person of ordinary skill in the art and is not to be limited to a special or customized meaning. The term specifically may refer, without limitation, to the automatically adjusted exposure parameter value(s) of a camera in order to control exposure or how much light is let in to an image. Exposure can, e.g., be regulated by adjusting exposure parameter values such as one or more of sensor gain, shutter speed or exposure time, lens aperture, or by adjusting any other operation of a camera for changing an exposure parameter value of the camera. Sensor gain may, e.g., be defined as a digital camera setting that controls the amplification of the signal from one or more of the camera sensors. Amplification of the signal may be controlled individually for each sensor or for a subset of sensors of the camera when adjusting sensor gain. Alternatively, amplification of the signal may be controlled uniformly for all sensors of the camera when adjusting sensor gain. Increasing the gain amplifies the signal by increasing the ratio of the analog-to-digital units (ADUs) to electrons acquired on the sensor. The result is that increasing gain increases the apparent brightness of an image. Shutter speed or exposure time may, e.g., be defined as the length of time the camera shutter is open, exposing light onto the camera sensor. Lens aperture may refer to a hole or an opening that limits the amount of light that can pass through. While for some mobile devices, exposure parameter values may be set manually by the user or by a third party app downloaded to the smartphone, many smartphones, especially cheaper ones, do not allow the user or a third party/their app to freely adjust exposure parameter values like sensor gain, shutter speed (exposure time), or lens aperture. They only offer automatic (“auto”) exposure settings (“auto-exposure mode”). Such automatic exposure settings, e.g., may try to take the image such that the integral intensity or brightness of the image is in accordance with predefined values.

When auto-exposure settings are determined or calculated, the result (the determined auto-exposure settings) should only be influenced by the scene or by a part of the scene to be imaged. An exposure metering area may be set to determine which part of the scene, i.e. which sub-scene, or whether the entire scene in the field of view of the camera is used by the camera to determine or calculate the auto-exposure settings. However, smartphones with camera systems exist which let a user or app (manufacturer) set an exposure metering area (or rather pretend to the user or app (manufacturer) that an exposure metering area may be set), but the actually used auto-exposure settings when an image is taken appear nevertheless to be influenced by parts of the scene outside of the sub-scene to which the exposure metering area was set by the user or app (manufacturer).

The term “mobile device” as used herein is a broad term and is to be given its ordinary and customary meaning to a person of ordinary skill in the art and is not to be limited to a special or customized meaning. The term specifically may refer, without limitation, to a mobile electronics device, more specifically to a mobile communication device such as a cell phone or a smartphone. Additionally or alternatively, as will be outlined in further detail below, the mobile device may also refer to a tablet computer or another type of portable computer having at least one camera.

The term “camera” as used herein is a broad term and is to be given its ordinary and customary meaning to a person of ordinary skill in the art and is not to be limited to a special or customized meaning. The term specifically may refer, without limitation, to a device having at least one imaging element configured for recording or capturing spatially resolved one-dimensional, two-dimensional or even three-dimensional optical data or information. The camera may specifically comprise one or more imaging devices, such as camera chips or imaging chips, e.g. CCD and/or CMOS chips. The camera, in particular the imaging device, may comprise a one-dimensional or two-dimensional array of image sensors, such as pixels. As an example, the camera may comprise at least 10 pixels in at least one dimension, such as at least 10 pixels in each dimension. It shall be noted, however, that other cameras are also feasible. The camera, besides at least one camera chip or imaging chip, may comprise further elements, such as one or more optical elements, e.g. one or more lenses. As an example, the camera may be a fix-focus camera, having at least one lens which is fixedly adjusted with respect to the camera. Alternatively, however, the camera may also comprise one or more variable lenses which may be adjusted, automatically or manually. The camera may also comprise a diaphragm for controlling the aperture (whereby the amount of light that reaches the camera sensor may be controlled). The diaphragm functions much like the iris of the eye-it controls the effective diameter of the lens opening (called the aperture). The camera may further comprise an ambient light sensor for measuring light reflected by a scene to be captured in an image.

The present disclosure specifically shall be applicable to cameras as usually used in mobile applications such as notebook computers, tablets or, specifically, cell phones such as smartphones. Thus, specifically, the camera may be part of a mobile device which, besides the at least one camera, comprises one or more data processing devices such as one or more data processors. Other cameras, however, are feasible.

The camera specifically may be a color camera. Thus, such as for each pixel, color information may be provided or generated, such as color values for three colors R, G, B. A larger number of color values is also feasible, such as four color values for each pixel, for example R, G, G, B. Color cameras are generally known to the skilled person. Thus, as an example, the camera chip may consist of a plurality of three or more different color sensors each, such as color recording pixels like one pixel for red (R), one pixel for green (G) and one pixel for blue (B). For each of the pixels, such as for R, G, B, values may be recorded by the pixels, such as digital values in the range of 0 to 255, depending on the intensity of the respective color. Instead of using color triples such as R, G, B, as an example, quadruples may be used, such as R, G, G, B. The color sensitivities of the pixels may be generated by color filters or by appropriate intrinsic sensitivities of the sensor elements used in the camera pixels. These techniques are generally known to the skilled person. For example, gain can be a digital camera setting that controls the amplification of the signal from the camera sensor.

As outlined above, steps a1) and b1) comprise receiving first and second exposure metering area settings to cover a first sub-scene of a first scene in the field of view of the camera and to cover a second sub-scene of a second scene or the entire second scene in the field of view of the camera, respectively.

Receiving exposure metering area settings in particular may refer to setting an exposure metering area. The exposure metering area settings may be received by a camera system of the mobile device. The term “exposure metering area” as used herein is a broad term and is to be given its ordinary and customary meaning to a person of ordinary skill in the art and is not to be limited to a special or customized meaning. The term specifically may refer, without limitation, to the area that should be considered by an auto-exposure algorithm to automatically determine or calculate and/or manipulate exposure parameter value(s) of the camera in such a way that the image is captured in a predetermined manner. By setting the exposure metering area it should be determined which part of a scene in the field of view of the camera is considered for determining the auto-exposure settings. Determining, e.g. by calculating, auto-exposure settings can then be defined generally as running any algorithm that automatically calculates and/or manipulates one or more of the exposure parameter value(s) of the camera in such a way that the image is captured in a predetermined manner. For example, there may be a predetermined optimum average brightness value for a given scene that the camera will try to achieve by adjusting one or more of the camera's exposure parameter values. Some auto-exposure algorithms, e.g., calculate and/or manipulate the exposure parameter values, e.g., the camera's gain, such that a mean, median, or weighted value of the image's brightness will equal a predetermined optimum brightness value.

The exposure metering area may be set to cover the scene (i.e. the entire scene) in the field of view of the camera or just a part of the scene in the field of view of the camera, i.e. a sub-scene. In the first case, the set exposure metering area thus corresponds to the entire scene in the field of view of the camera. In the second case, the set exposure metering area only corresponds to part of the scene in the field of view of the camera. If auto-exposure settings are determined based on the entire scene in the field of view of the camera (100 % of the scene in the field of view of the camera), this may be termed “whole-frame metering”. If auto-exposure settings are determined based on just a part, i.e. a sub-scene (e.g., up to 10% or up to 20% or up to 30%) of the scene in the field of view of the camera, such as the part in the center of the field of view of the camera this may be termed “center spot metering” or a part of the scene in the field of the view of the camera that is off the center this may be termed “off-center spot metering”, e.g., around an off-centered selected focus point. Determining, e.g. by calculating, auto-exposure settings based on the part of a scene in the set exposure metering area in this case means to determine the auto-exposure settings based on that part of the scene in the field of view of the camera only and not based on other parts of the scene in the field of view.

Receiving the exposure metering area settings to cover a first sub-scene of a first scene in the field of view of the camera may be initiated by user action or may automatically be initiated, e.g. once the presence of at least one target object within a field of view and/or within a predetermined part of the field of view of the camera is automatically detected. In contrast, the actual determination of the auto-exposure settings by the mobile device may be a device-specific process unknown to the manufacturer of a measurement application.

The first sub-scene may constitute 10-70% or 20-60% or 30-50% of the first scene (or in other words: of the entire first scene). The second sub-scene may constitute 10-70% or 20-60% or 30-50% of the second scene (or in other words: of the entire second scene).

As outlined above, steps a2) and b2) comprise capturing a first image of the first scene and capturing a second image of the second scene, respectively.

The term “image” as used herein is a broad term and is to be given its ordinary and customary meaning to a person of ordinary skill in the art and is not to be limited to a special or customized meaning. The term may specifically refer, without limitation, to a set of spatially resolved optical data. Specifically, the term may relate to data recorded by using a camera, such as a plurality of electronic readings from the imaging device, such as the pixels of the camera chip.

The term “capturing an image” as used herein is a broad term and is to be given its ordinary and customary meaning to a person of ordinary skill in the art and is not to be limited to a special or customized meaning. The term specifically may refer, without limitation, to one or more of imaging, image recording, image acquisition, image capturing. The term “capturing an image” may comprise capturing at least one single image, i.e., one image and/or a plurality of images such as a sequence of images. For example, the capturing of the image may comprise recording continuously a sequence of images such as a video or a movie. The capturing of the image may be initiated by user action or may automatically be initiated, e.g., once the presence of at least one target object within the field (or a predetermined part of the field) of view of the camera is automatically detected. These automatic image acquisition techniques are known, e.g., in the field of automatic barcode readers, such as from automatic barcode reading apps. The capturing of the images may take place, as an example, by acquiring a stream or “live stream” of images with the camera, wherein one or more of the images, automatically or by user interaction such as pushing a button, are stored and one of them is used as the at least one image. The image acquisition may be supported by a processor of the mobile device. Storing of the images may take place in a data storage device of the mobile device.

As outlined above, steps a3) and b3) comprise determining a first intensity parameter from the first image and determining a second intensity parameter from the second image, respectively.

The term “intensity parameter” as used herein is a broad term and is to be given its ordinary and customary meaning to a person of ordinary skill in the art and is not to be limited to a special or customized meaning. The term specifically may refer, without limitation, to a statistical value descriptive of the intensity values from a certain set or substantially all of the pixels of an image; i.e., the entire image. The intensity parameter may, e.g., be determined as the maximum, minimum, median or averaged intensity value of those pixels of an image corresponding to a certain sub-scene or corresponding to a target object in the image or of all pixels of the image. The intensity values may, e.g., be digital values in the range of 0 to 255.

As outlined above, step c) comprises determining a suitability parameter by using the first intensity parameter and the second intensity parameter.

The term “suitability parameter” as used herein is a broad term and is to be given its ordinary and customary meaning to a person of ordinary skill in the art and is not to be limited to a special or customized meaning. The term specifically may refer, without limitation, to the outcome of a statistical analysis, comparison of values, i.e., a first value being larger, smaller or equal to a second value, and/or numerical operation such as addition, subtraction, multiplication and division of two values or parameters.

In one embodiment, the suitability parameter being outside a predefined suitability range indicates non-suitability of the auto-exposure settings of the mobile device for performing a color based measurement and initiates a non-suitability action.

In one embodiment, the suitability parameter being inside a predefined suitability range indicates suitability of the auto-exposure setting of the mobile device for performing a color based measurement and initiates a suitability action.

The suitability parameter being inside a predefined suitability range means, e.g., the suitability parameter is within a certain suitability range defined by a lower and an upper predefined suitability value, respectively, or is smaller than or equal to a predefined first suitability value or larger than or equal to a predefined second suitability value. The suitability parameter being outside a predefined suitability range means, e.g., the suitability parameter is outside a certain suitability range defined by a lower and an upper predefined suitability value, respectively, or is higher than a predefined first suitability value or smaller than a predefined second suitability value. The comparison to the predefined value or values results in either a suitability or non-suitability of the auto-exposure settings of the mobile device for performing a color based measurement. The suitability parameter being outside a predefined suitability range indicates non-suitability and initiates a non-suitability action. The suitability parameter being inside a predefined suitability range indicates suitability and initiates a suitability action.

For the suitability evaluation of the auto-exposure settings of the mobile device for performing a color based measurement it is required that either the second exposure metering area settings are different from the first exposure metering area settings, or the second scene is different from the first scene.

In one embodiment, the non-suitability action comprises providing a user warning notification and/or blocking an analytical measurement based on a color formation reaction with the mobile device.

The term “user warning notification” as used herein is a broad term and is to be given its ordinary and customary meaning to a person of ordinary skill in the art and is not to be limited to a special or customized meaning. For example, a user warning notification is a message on a display, a vibration or acoustic signal. Preferably, the user warning notification is performed on or by the mobile device to inform the user that the mobile device is not suitable for performing a color based measurement, such as an analytical measurement.

In one embodiment, the user warning notification comprises a notification of under-and/or overexposed image area(s) corresponding to a target object during the performance of a color-based measurement and optionally comprising a prompt to choose a lighter or darker background for placing the target object thereon than the one chosen for said image with under-and/or overexposed image area(s) when taking another image.

The term “blocking a color based measurement” as used herein is a broad term and is to be given its ordinary and customary meaning to a person of ordinary skill in the art and is not to be limited to a special or customized meaning. For example, blocking a color based measurement means to not allow a color based measurement of an analyte or to prevent the display of a result of a color based measurement on the display of a smartphone with a measurement app downloaded to the smartphone.

In one embodiment, the suitability action comprises unlocking an analytical measurement based on a color formation reaction with the mobile device. In one embodiment, the suitability action comprises a notification to the user informing about the suitability.

The term “unlocking a color based measurement” as used herein is a broad term and is to be given its ordinary and customary meaning to a person of ordinary skill in the art and is not to be limited to a special or customized meaning. For example, unlocking a color based measurement means to allow a color based measurement of an analyte or to allow the display of a result of a color based measurement on the display of a smartphone with a measurement app downloaded to the smartphone. In one embodiment, the suitability parameter is the ratio of the first and second intensity parameter.

The mobile device may comprise a display. The camera and the display of the mobile device may both be positioned on the same side of the mobile device (in this case the camera may be termed a front camera) or on opposite sides of the mobile device (in this case the camera may be termed a back camera). Specifically, the camera may be a back camera. As one example, the mobile device may comprise a display, and the method of the present disclosure may comprise providing a notification on the display, to inform the user about the non-suitability or suitability, to provide a warning, to provide visual guidance for positioning the camera, or to indicate access changes to an application or access changes to certain functionalities of an application, wherein the access changes are, for example, related to user-access, user access to functionalities or access between different applications, e.g., access to information exchange.

In one embodiment, the first scene comprises or consists of the first sub-scene and a first background sub-scene (also termed first background scene herein), and the second scene comprises or consists of the second sub-scene and a second background sub-scene (also termed second background scene herein). The sub-scene and the background scene usually do not overlap. In one embodiment, the first scene comprises or consists of a first sub-scene and a first background scene, wherein the first sub-scene and the first background have a maximum overlap of 15%; the second scene comprises or consists of a second sub-scene and a second background scene, wherein the second sub-scene and the second background scene have a maximum overlap of 15 %.

In a further embodiment, the first scene comprises or consists of a first sub-scene and a first background scene, wherein the first sub-scene and the first background have a maximum overlap of 10 or 5% or they do not overlap at all; the second scene comprises or consists of a second sub-scene and a second background scene, wherein the second sub-scene and the second background scene have a maximum overlap of 10 or 5 % or they do not overlap at all.

In one embodiment, the first and second exposure metering area settings are identical, the first and second sub-scene are identical and comprising a target object while the first and second background sub-scene are different. A sub-scene comprising a target object may mean that the target object completely fills the sub-scene or that it fills the sub-scene to, e.g., at least 80 or 90%.

In a second embodiment, the first and second scene are identical while the first and second exposure metering area settings are different in that the first exposure metering area settings are to cover the first sub-scene comprising a target object, the second exposure metering area settings are to cover the entire second scene.

In a third embodiment, the first and second scene are identical while the first and second exposure metering area settings differ from each other to cover different sub-scenes thereof.

Different with respect of scenes (or sub-scenes or background scenes) herein, e.g., means that the two scenes (or two sub-scenes or two background scenes) do not overlap at all, or there is a maximum overlap of 15 or 10 or 5 %. Identical with respect to scenes (or sub-scenes or background scenes) herein, e.g., means that two scenes (or two sub-scenes or two background scenes) overlap to at least 85 or 90 or 95 or 100 %. Different with respect of exposure metering area settings means that two set exposure metering areas do not overlap at all, or there is a maximum overlap of 15 or 10 or 5 %. Identical with respect to exposure metering area settings herein, e.g., means that two set exposure metering areas overlap to at least 85 or 90 or 95 or 100 %.

The first and second intensity parameter can be determined as the intensity of the partial first and partial second image corresponding to the first and second sub-scene (I_{S1}, I_{S2}), respectively, as the intensity of the partial first and partial second image corresponding to a target object (I_{T1}, I_{T2}), respectively, or as the intensity of the first and second image (i.e. the entire first and second image) (I_{Tot1}, I_{Tot2}), respectively.

The intensity of a partial image corresponding to a sub-scene or corresponding to a target object may be the maximum, minimum, median or averaged intensity value of those pixels of an image corresponding to the sub-scene or corresponding to the target object in the image. The intensity of an image (i.e. the entire image) may be the maximum, minimum, median or averaged intensity value of all or substantially all of the pixels of the image.

The suitability parameter is, e.g., the ratio of the first and second intensity parameter determined as the intensity of the partial first and second image corresponding to the first and second sub-scene, respectively: I_{S1}/I_{S2.} In another example, the suitability parameter is, e.g., the ratio of the first and second intensity parameter determined as the intensity of the partial first and second image corresponding to the target object in the first and second image, respectively: I_{T1}/I_{T2}.

In one example, the predefined suitability range is from 0.83 to 1.2. If, e.g., I_{S1}/I_{S2} or I_{T1}/I_{T2} thus is ≥0.83 and ≤1.2 the suitability parameter is inside the predefined suitability range in this example. In another example, the predefined suitability range is from 0.90 to 1.1 or 0.95 to 1.05. If, e.g., I_{S1}/I_{S2} or I_{T1}/I_{T2} thus is ≥0.9 and ≤1.1 or ≥0.95 and ≤1.05 the suitability parameter is inside the predefined suitability range in this example.

In another example, the suitability parameter is, e.g., the ratio of the first and second intensity parameter determined as the intensity of the entire first and second image, respectively: I_{Tot1}/I_{Tot2}.

In one example, the predefined suitability range consists of two predefined partial suitability ranges that are not overlapping. In this case, a suitability parameter being inside the predefined suitability range, e.g., means that the suitability parameter is either in the first predefined partial suitability range or in the second predefined partial suitability range. The first predefined partial suitability range is, e.g., from 0 to <0.95 or from 0 to <0.9. The second predefined partial suitability range, e.g., covers any value >1.05 or >1.1 or is from >1.05 to <10, or from >1.1 to <5. If, e.g., I_{Tot1}/I_{Tot2} is ≥0 and <0.95, or >0 and <0.9 it is inside the first predefined partial suitability range in one embodiment. If, e.g., I_{Tot1}/I_{Tot2} is >1.05, or >1.1 it is inside the second predefined partial suitability range in one embodiment.

In one embodiment, the method comprises:
a4) determining a third intensity parameter from the first image; and/or
b4) determining a fourth intensity parameter from the first or second image; and determining an admissibility parameter by using the third intensity parameter and the fourth intensity parameter, and/or by using the third intensity parameter and the first intensity parameter,
wherein the suitability action is only initiated if the admissibility parameter is inside a predefined admissibility range.

The admissibility parameter is, e.g., based on the ratio of the third and fourth intensity parameter, and/or the ratio of the third and first intensity parameter.

The term “admissibility parameter” as used herein is a broad term and is to be given its ordinary and customary meaning to a person of ordinary skill in the art and is not to be limited to a special or customized meaning. The term specifically may refer, without limitation, to the outcome of a statistical analysis, comparison of values, i.e., a first value being larger, smaller or equal to a second value, and/or numerical operation such as addition, subtraction, multiplication and division of two values or parameters.

The admissibility parameter is compared to a predefined admissibility range, i.e., the admissibility parameter is within a certain admissibility range defined by a lower and an upper value or is smaller than or equal to a first predefined admissibility value or larger than or equal to a second predefined admissibility value.

The third intensity parameter can, e.g., be determined as the intensity of the partial first image corresponding to the first background scene (I_{B1}), or as the intensity of the partial first image corresponding to the first sub-scene (I_{S1}). The fourth intensity parameter can, e.g., be determined as the intensity of the partial first or second image corresponding to the second background scene (I_{B2}), or as the intensity of the partial first or second image corresponding to the first or second sub-scene (I_{S1}, I_{S2}), respectively.

The admissibility parameter is, e.g., the ratio of the third and fourth intensity parameter determined as the intensity of the partial first and second image corresponding to the first and second background scene, respectively: I_{B1}/I_{B2.} As an example, the predefined admissibility range is from 0 to 0.77 or 0 to 0.71 or 0 to 0.67 and/or >1.3 or >1.4 or >1.5.

In one embodiment, the first and the second scene comprise at least part of a target object.

The term “target object” as used herein is a broad term and is to be given its ordinary and customary meaning to a person of ordinary skill in the art and is not to be limited to a special or customized meaning. The term specifically may refer, without limitation, to a recognizable object, a selected object, an object comprising a detectable identifier and/or position marker. In one embodiment, the target object is a color reference card.

The term “color reference card” as used herein is a broad term and is to be given its ordinary and customary meaning to a person of ordinary skill in the art and is not to be limited to a special or customized meaning. The term specifically may refer, without limitation, to an arbitrary item having, disposed therein or disposed thereon, such as on at least one surface, a plurality of different color reference fields having known color properties or optical properties, such as having a plurality of colored fields having known reference color values, and further one or more gray background fields having defined gray levels. As an example, the color reference card may be a flat card comprising at least one substrate having, on at least one surface and/or disposed therein, the plurality of color reference fields having known color coordinates and one or more gray background fields having known gray levels. The color reference card may also comprise one or more further gray fields.

The substrate, specifically, may have a flat surface comprising the plurality of color reference fields, and the one or more gray background fields, and optionally (additional) gray fields disposed thereon. The substrate, as an example, may be or may comprise one or more of a paper substrate, a cardboard substrate, a plastic substrate, a ceramic substrate or a metal substrate. Laminate substrates are also possible. The substrate, as an example, may be sheet-like or flexible. It shall be noted, however, that the substrate may also be implemented into an article of use, such as into a wall of a box, a vile, a container, a medical consumable, such as a test strip, or the like. Thus, the color reference card may also fully or partially be integrated into the optical test strip. Thus, the at least one image of at least a part of the color reference card may fully or partially comprise an image of at least part of the optical test strip having at least one reagent test field.

The term “optical test strip” as used herein is a broad term and is to be given its ordinary and customary meaning to a person of ordinary skill in the art and is not to be limited to a special or customized meaning. The term specifically may refer, without limitation, to a strip shaped element or device configured for performing a color-change detection reaction. The optical test strip may also just be referred to as test strip herein, wherein these terms may refer to the same element. The optical test strip may particularly have a reagent test field containing at least one test chemical for detecting at least one analyte. The optical test strip, as an example, may comprise at least one substrate, such as at least one carrier, with the at least one reagent test field applied thereto or integrated therein. In particular, the optical test strip may further comprise at least one white area, such as a white field, specifically in a proximity to the test field, for example enclosing or surrounding the test field. The white area may be a separate field independently arranged on the substrate or carrier. However, additionally or alternatively, the substrate or carrier itself may be or may comprise the white area. The at least one carrier may be strip-shaped, thereby providing the basic form of the test strip. These test strips are generally widely in use and available. One test strip may carry a single test field or a plurality of test fields having identical or different test chemicals comprised therein.

As further used herein, the term “reagent test field”, also referred to as “test field”, is a broad term and is to be given its ordinary and customary meaning to a person of ordinary skill in the art and is not to be limited to a special or customized meaning. The term specifically may refer, without limitation, to a coherent amount of the test chemical, such as to a field or a region, e.g., a field of round, polygonal or rectangular shape, having one or more layers of material, with at least one layer of the test field having the test chemical comprised therein.

The optical test strip may be placed on top of the color reference card, and/or the color reference card may comprise one or more windows, wherein the color reference card, with the one or more windows, is placed on top of the optical test strip such that the reagent test region is visible through the window.

In one embodiment, the color reference card comprises at least one position marker.

The at least one position marker, as an example, may be or may comprise particularly an ArUco code or the like. Specifically, the at least one position marker may be arranged in at least one corner of the color reference card. Thus, the mobile device may be configured for detecting and/or reading the marker, specifically by optically detecting the marker (e.g., in the first scene and/or in the second scene of step a1) and b1), respectively; or on the at least one first image and/or on the at least one second image captured in steps a2) and b2), respectively), and optionally retrieving information from the marker, such as information on the position and/or orientation of the color reference card in relation to the camera. As an example, position markers can also be located in each corner of the color reference card. The color reference card may include other marker(s) including further information or the position marker may additionally include further information. Such further information may include at least one of: an identifier for identifying the color reference card and/or the type of the color reference card, such as at least one of a label, a barcode or a QR-code; a specifier specifying details of the color reference card, such as reference color values, gray background and/or further gray values or the like, such as by using at least one of a label, a barcode or a QR-code.

Additionally or alternatively, the test strip may comprise at least one position marker.

The mobile device may be configured for detecting and/or reading the one or more position marker(s), specifically by optically detecting the marker(s). Thus, the mobile device may be configured for detecting and/or reading the marker(s) specifically by optically detecting the marker(s), and optionally retrieving information therefrom, such as information on the type, the properties or the position of the color reference card relative to the camera. Thereby, the mobile device may, e.g., automatically determine that the camera is in a defined position with respect to the color reference card.

The color reference card may contain at least one positioning element for positioning the optical test strip and/or the reagent test field. Specifically, the color reference card may comprise at least one window element through which the reagent test field is visible when the test strip is placed behind the color reference card. For example, the window element may be a cut-out portion of the color reference card. The window element may specifically be configured for keeping the optical test strip and, thus, the reagent test field comprised by the optical test strip, in the defined position with respect to the color reference card. The reagent test field may in particular be in a defined position with respect to the color reference card during one or both of determining the exposure parameters for the camera and capturing the at least one image. Thereby optimal performance, in terms of accuracy of the measurement results achieved may be ensured.

The mobile device may automatically determine the presence of the color reference card and/or a certain position thereof within a field of view and/or within a predetermined part of the field of view of the camera, such as the set exposure metering area, based on the position marker(s). Additionally, or alternatively, the capturing of the at least one image may be initiated automatically, in particular once the presence of at least one object within a field of view and/or within a predetermined part of the field of view of the camera is automatically detected, the object comprising at least part of the optical test strip and/or at least part of the color reference card.

The target object, e.g., comprises or consists of at least 60, 70, 80 or 90% of the color reference card.

The at least one analyte, as an example, may be or may comprise one or more specific chemical compounds and/or other parameters. As an example, one or more analytes may be determined which take part in metabolism, such as blood glucose. Additionally or alternatively, other types of analytes or parameters may be determined, e.g. a pH value.

The method may further comprise after suitability has been determined: determining the concentration of an analyte in a sample based on an image of a test field after application of the sample, the image having been captured with the mobile device.

In a further aspect of the present disclosure, a mobile device is disclosed, having at least one camera for capturing an image representative of a scene, the mobile device being configured for performing the method for evaluating the suitability of auto-exposure settings of the mobile device for performing a color based measurement as described above.

The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the mobile device, computer or system. As an example, the processor may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) or the like.

In a further aspect of the disclosure, a computer program is disclosed, the computer program comprising instructions which, when the program is executed by a mobile device having a camera, cause the processor of the mobile device to perform the method for evaluating the suitability of auto-exposure settings of the mobile device for performing a color based measurement as described above.

In a further aspect of the present disclosure, a kit is disclosed, the kit comprising at least one mobile device according to the present disclosure, such as according to any one of the embodiments disclosed above and/or according to any one of the embodiments disclosed in further detail below, further comprising at least one optical test strip having at least one reagent test field, and at least one color reference card. The term “kit” as used herein is a broad term and is to be given its ordinary and customary meaning to a person of ordinary skill in the art and is not to be limited to a special or customized meaning. The term specifically may refer, without limitation, to a combination of at least two items which might as an example, may be supplied conjointly in a package, which may interact in order to fulfill at least one common purpose.

In a further aspect of the disclosure, a computer program is disclosed, the computer program comprising instructions which, when the program is executed by a mobile device having a camera cause the processor of the mobile device to perform steps a1), a3), b1), b3) and c) (and optionally also steps a4) and b4) of the method for evaluating the suitability of auto-exposure settings of a mobile device.

In a further aspect of the disclosure, a computer-readable storage medium is disclosed, the computer-readable storage medium comprising instructions which, when the program is executed by a mobile device having a camera cause the processor of the mobile device to perform steps a1), a3), b1, b3) and c) (and optionally also steps a) and b4) of the method for evaluating the suitability of auto-exposure settings of a mobile device.

As used herein, the term “computer-readable storage medium” specifically may refer to a non-transitory data storage means, such as a hardware storage medium having stored there-on computer-executable instructions. The computer-readable data carrier or storage medium specifically may be or may comprise a storage medium such as a random-access memory (RAM) and/or a read-only memory (ROM).

The computer program may also be embodied as a computer program product. As used herein, a computer program product may refer to the program as a tradable product. The product may generally exist in an arbitrary format, such as in a paper format, or on a computer-readable data carrier and/or on a computer-readable storage medium. Specifically, the computer program product may be distributed over a data network.

The methods and devices according to the present disclosure enable the use of a greater variety of models of mobile devices for an analytical measurement. In particular, they allow to determine suitability of a mobile device (or a type of mobile device) for performing a color based measurement, even when the mobile device (or that type of mobile device) has not been tested in a laboratory setting, by, e.g., the manufacturer of an app to be downloaded to such mobile device or type of mobile device for performing a color based measurement therewith. More particularly, they allow to identify mobile devices with auto-exposure settings as unsuitable or as suitable to ensure an accurate analytical measurement.

### BRIEF DESCRIPTION OF THE DRAWINGS

Further optional features and embodiments will be disclosed in more detail in the subsequent description of embodiments. The respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1a, 1b, 1c and 1d show a first embodiment with identical first and second sub-scenes, differing first and second background scenes, and identical first and second metering area settings;

FIGS. 2a, 2b, 2c and 2d show a second embodiment with identical first and second scenes, and differing first and second exposure metering area settings;

FIGS. 3a, 3b, 3c and 3d show a third embodiment with identical first and second scenes, and differing first and second exposure metering area settings; and

FIG. 4 shows a flow chart of an embodiment of a method for evaluating the suitability of auto-exposure settings of a mobile device for performing a color based measurement.

### DETAILED DESCRIPTION

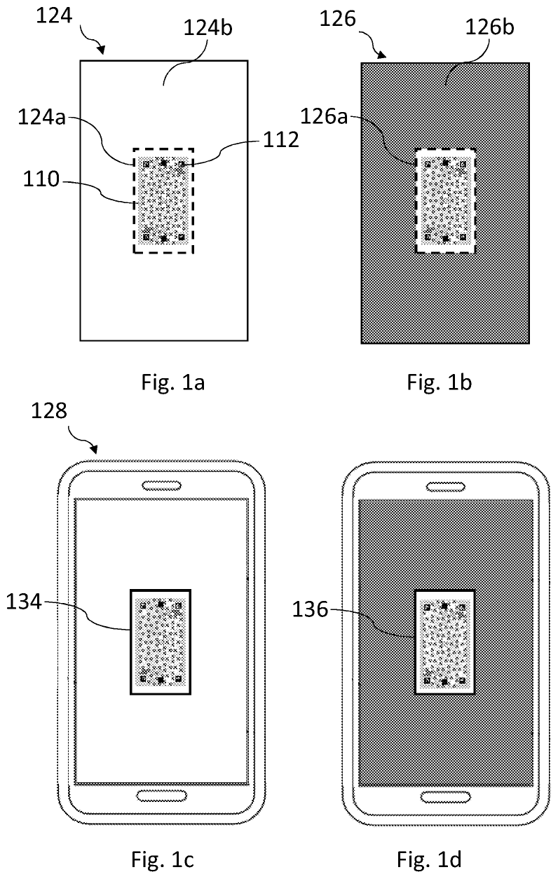

In FIG. 1a a first scene 124 is shown comprising a first sub-scene 124a and a light first background scene 124b. The first sub-scene 124a comprises a color reference card 110 as a target object which fills the first sub-scene 124a almost entirely. The color reference card 110 comprises position markers 112. FIG. 1b shows a second scene 126 comprising a second sub-scene 126a and a dark second background scene 126b. The second scene 126 is different from the first scene 124 in that the second background scene 126b is dark while the first background scene 124b is light. In contrast, the second sub-scene 126a is identical to the first sub-scene 124a. FIG. 1c shows a mobile device 128 comprising a camera. On the display of the mobile device 128 a scene in the field of view of the camera is seen which is first scene 124. First exposure metering area settings 134 are received to cover the first sub-scene 124a of the first scene 124 in the field of view of the camera. A first image of the first scene 124 is then captured with the camera of the mobile device 128. FIG. 1d again shows the mobile device 128. On the display of the mobile device 128 in FIG. 1d, however, a different scene in the field of view of the camera is seen which is second scene 126. Second exposure metering area settings 136 are received to cover the second sub-scene 126a of the second scene 126 in the field of view of the camera. The second exposure metering area settings 136 are identical to the first exposure metering area settings 134. A second image of the second scene 126 is then captured with the camera of the mobile device 128.

Ideally, suitability is indicated if the average or median intensity of the partial first image corresponding to the first sub-scene 124a (or corresponding to the target object 110) and the average or median intensity of the partial second image corresponding to the second sub-scene 126a (or corresponding to the target object 110) are the same for both images, i.e. I_{S1}/I_{S2}=1 (or I_{T1}/I_{T2}=1). However, suitability may also be indicated if 0.83≤I_{S1}/I_{S2}≤1.2 or 0.83≤I_{T1}/I_{T2}≤1.2 This allows for some tolerance. In contrast, if I_{S1}/I_{S2} (or I_{T1}/I_{T2}) is outside the range of 0.83 to 1.2, the change in intensity of the partial first image corresponding to the first sub-scene 124 a (or corresponding to the target object 110) to the intensity of the partial second image corresponding to the second sub-scene 126a (or corresponding to the target object 110) is larger than what would be expected if auto-exposure settings would indeed be based only on the set first and second exposure metering area, respectively. If I_{S1}/I_{S2} (or I_{T1}/I_{T2}) is outside the range of 0.83 to 1.2 non-suitability of the auto-exposure settings of the mobile device 128 for performing a color based measurement may thus be indicated.

Furthermore, an admissibility parameter may be determined as the ratio of the average or median intensity of the partial first/second image corresponding to the first/second background scene, respectively, i.e., I_{B1}/I_{B2}. Suitability of the auto-exposure settings of the mobile device 128 for performing a color based measurement may then only be indicated if I_{B1}/I_{B2}<0.77 or <0.71 or <0.67 or I_{B1}/I_{B2}>1.30 or >1.40 or >1.50. In this case the second background scene 126b would be sufficiently darker/brighter than the first background scene 124b to ensure that it was detectable if auto-exposure settings were not only influenced by the set first and second exposure metering area.

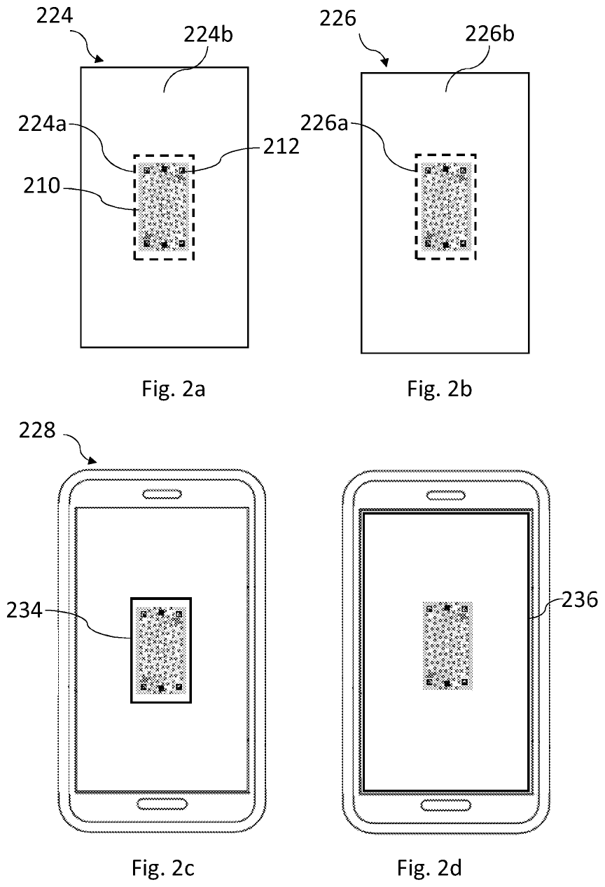

In FIG. 2a a first scene 224 is shown comprising a first sub-scene 224a and a first background scene 224b. The first sub-scene 224a comprises a color reference card 110 as a target object which fills the first sub-scene 224a almost entirely. The color reference card 210 comprises position markers 212. FIG. 2b shows a second scene 226 comprising a second sub-scene 226a and a second background scene 226b. The second scene 226 is the same, or in other words is identical to the first scene 224, i.e. the first and second sub-scene 224a and 226a are identical and the first and second background scene 224b and 226b are identical. FIG. 2c shows a mobile device 228 comprising a camera. On the display of the mobile device 228 a scene in the field of view of the camera is seen which is first scene 224. First exposure metering area settings 234 are received to cover the first sub-scene 224a of the first scene 224 in the field of view of the camera. A first image of the first scene 224 is then captured with the camera of the mobile device 228. FIG. 2d again shows the mobile device 228. On the display of the mobile device 228 in FIG. 2d, the same scene in the field of view of the camera is seen as in FIG. 2c, but it is referenced as second scene 226 in FIG. 2b. Second exposure metering area settings 236 are received to cover the entire scene in the field of view of the camera, i.e. the second scene 226. The second exposure metering area settings 236 are thus different to the first exposure metering area settings 234. A second image of the second scene 226 is then captured with the camera of the mobile device 228.

Suitability may be indicated if the average or median intensity of the partial first image corresponding to the first sub-scene 224a (or corresponding to the target object 210) and the average or median intensity of the partial second image corresponding to the second sub-scene 226a (or corresponding to the target object 210) is sufficiently different, i.e., e.g., either I_{S1}/I_{S2} (or I_{T1}/I_{T2})≤0.95 or ≤0.9, or I_{S1}/I_{S2} (or I_{T1}/I_{T2})≥1.05 or ≥1.1. In contrast, if, e.g. 0.95>I_{S1}/I_{S2}<1.05 (or 0.9>I_{T1}/I_{T2}<1.1), the change in intensity of the partial first image corresponding to the first sub-scene 224a (or corresponding to the target object 210) to the intensity of the partial second image corresponding to the second sub-scene 226a (or corresponding to the target object 210) is smaller than what would be expected if auto-exposure settings were indeed be based only on the set first and second exposure metering area, respectively. If 0.95>I_{S1}/I_{S2}<1.05 (or 0.9>I_{T1}/I_{T2}<1.1), non-suitability of the auto-exposure settings of the mobile device 228 for performing a color based measurement may thus be indicated.

Furthermore, an admissibility parameter may be determined as the ratio of the average or median intensity of the partial first image corresponding to the first background scene 224b and the intensity of the partial first image corresponding to the first sub-scene 224a or to the target object 210, i.e. I_{B1}/I_{S1} or I_{B1}/I_{T1}. Suitability of the auto-exposure settings of the mobile device 228 for performing a color based measurement may then only be indicated if I_{B1}/I_{S1} or I_{B1}/I_{T1}<0.77 or <0.71 or <0.67, or I_{B1}/I_{S1} or I_{B1}/I_{T1}>1.30 or >1.40 or >1.50. In this case the first background scene 224b would be sufficiently brighter/darker than the first sub-scene 224a or the target object 210 to ensure that it was detectable if auto-exposure settings were not only influenced by the set first and second exposure metering area.

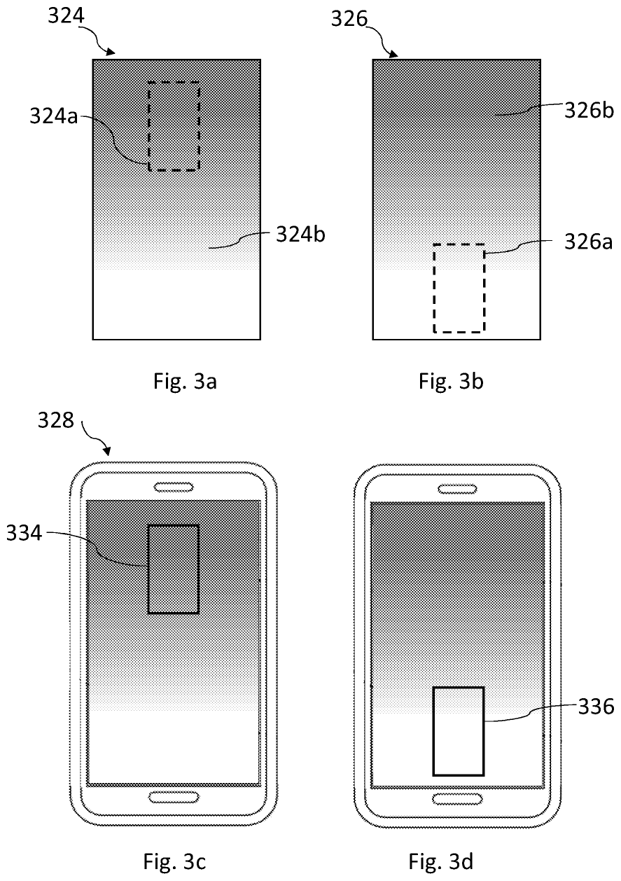

In FIG. 3a a first scene 324 is shown comprising a first sub-scene 324a and a first background scene 324b. This time, there is no target object in the scene. FIG. 3b shows a second scene 326 comprising a second sub-scene 326a and a second background scene 326b. The second scene 326 is the same, or in other words is identical to the first scene 324, however, the first and second sub-scene 324a and 326a are different as they correspond to different parts of the first and second scene, respectively. Accordingly, the first and second background scene 324b and 326b are also different. FIG. 3c shows a mobile device 328 comprising a camera. On the display of the mobile device 328 the scene in the field of view of the camera is seen which is first scene 324. First exposure metering area settings 334 are received to cover the first sub-scene 324a of the first scene 324 in the field of view of the camera. A first image of the first scene 324 is then captured with the camera of the mobile device 328. FIG. 3d again shows the mobile device 328. On the display of the mobile device 328 in FIG. 3d, the same scene in the field of view of the camera is seen which is this time referenced as second scene 326. Second exposure metering area settings 336 are received to cover the second sub-scene 336a which is different from first sub-scene 334a. While the size of first and second metering area is the same, the sub-scenes covered are different. The second exposure metering area settings 336 are thus different to the first exposure metering area settings 334. A second image of the second scene 326 is then captured with the camera of the mobile device 328.

Suitability may be indicated if the average or median intensity of the first image corresponding to the first scene 324 and the average or median intensity of the second image corresponding to the second scene 326 is sufficiently different, i.e., e.g., either I_{Tot1}/I_{Tot2}≤0.95 or ≤0.9, or I_{Tot1}/I_{Tot2}≥1.05 or ≥1.1. In contrast, if, e.g. 0.95>I_{Tot1}/I_{Tot2}<1.05 (or 0.9>I_{Tot1}/I_{Tot2}<1.1), the change in intensity of the first image corresponding to the first scene 324 to the intensity of the second image corresponding to the second scene 326 is smaller than what would be expected if auto-exposure settings were indeed be based only on the set first and second exposure metering area, respectively. If 0.95>I_{Tot1}/I_{Tot2}<1.05 (or 0.9>I_{Tot1}/I_{Tot2}<1.1), non-suitability of the auto-exposure settings of the mobile device 328 for performing a color based measurement may thus be indicated.

Furthermore, an admissibility parameter may be determined as the ratio of the average or median intensity of the partial first image corresponding to the first sub-scene 324a and the intensity of the partial second image corresponding to the second sub-scene 324b, i.e. I_{S1}/I_{S2}. Suitability of the auto-exposure settings of the mobile device 328 for performing a color based measurement may then only be indicated if I_{S1}/I_{S2}<0.77 or <0.71 or <0.67, or I_{S1}/I_{S2}>1.30 or >1.40 or >1.50. In this case the first sub-scene 324a would be sufficiently brighter/darker than the second sub-scene 326a to ensure that it was detectable if auto-exposure settings were not only influenced by the set first and second exposure metering area.

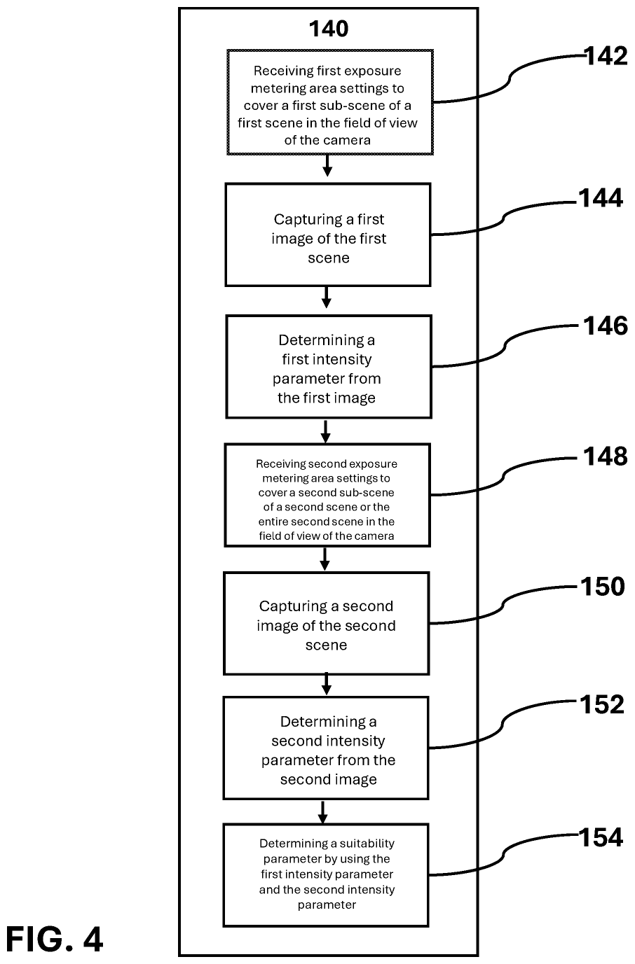

FIG. 4 shows a flow chart of an exemplary embodiment of a method 140 for evaluating the suitability of auto-exposure settings of a mobile device (128, 228, 328) for performing a color based measurement, the mobile device having at least one camera for capturing an image representative of a scene.

The method comprises:
a1) (denoted with reference number 142) receiving first exposure metering area settings (134,234,334) to cover a first sub-scene (124a, 224a, 324a) of a first scene (124, 224, 324) in the field of view of the camera;
a2) (denoted with reference number 144) capturing a first image of the first scene;
a3) (denoted with reference number 146) determining a first intensity parameter from the first image;
b1) (denoted with reference number 148) receiving second exposure metering area settings (136,236,336) to cover a second sub-scene (126a, 226a, 326a) of a second scene (126, 226, 326) or the entire second scene (126, 226, 326) in the field of view of the camera;
b2) (denoted with reference number 150) capturing a second image of the second scene;
b3) (denoted with reference number 152) determining a second intensity parameter from the second image;
c) (denoted with reference number 154) determining a suitability parameter by using the first intensity parameter and the second intensity parameter,
wherein the suitability parameter being outside a predefined suitability range indicates non-suitability of the auto-exposure settings of the mobile device for performing a color based measurement and initiates a non-suitability action; and/or
wherein the suitability parameter being inside a predefined suitability range indicates suitability of the auto-exposure setting of the mobile device for performing a color based measurement and initiates a suitability action, wherein either the second exposure metering area settings are different from the first exposure metering area settings, or the second scene is different from the first scene.

The method may comprise additional steps that are not listed.

In step a1) first exposure metering area settings 134, 234, 334 are received by the processor of the mobile device 128, 228, 328 to cover a first sub-scene (124a, 224a, 324a) of a first scene (124, 224, 324) in the field of view of the camera. The exposure metering area may particularly be set by an app (due to the app's program code executed by the processor of the mobile device) downloaded to the mobile device or by a user of the mobile device.

In step a2) a first image of the first scene is captured. The capturing of the first image may be initiated by user action or may automatically be initiated (after the first exposure metering area settings were received), e.g. once a target object such as a color reference card 110, 210 is detected in the field of view of the camera based on position markers 112, 212 of the color reference card. The image acquisition may be supported by the processor of the mobile device 128, 228, 328 and the storing of the image may take place in a data storage device of the mobile device. For capturing the first image, first auto-exposure settings may be used. The first auto-exposure settings may be determined based on the received first exposure metering area settings of step a1) or based on exposure metering area settings different therefrom. The mobile device may let a user or an app set a first exposure metering area (or rather pretend to the user or the app that a first exposure metering area may be set to be used for determining first auto-exposure settings only based thereon and not based on other parts of the scene in the field of view), i.e. certain first exposure metering area settings are received in step a1), but actually used first auto-exposure settings when capturing the image are nevertheless influenced by parts of the scene outside of the sub-scene or scene to which the exposure metering area was set by the user or the app. In this case, the actually used first exposure metering area for determining first auto-exposure settings of the device thus is different from the first exposure metering area set by the user or app.

In step a3) a first intensity parameter is determined from the first image. The first intensity parameter may, e.g., be determined as the maximum, minimum, median or averaged intensity value of those pixels of an image corresponding to the first sub-scene or corresponding to a target object in the first image or of all pixels of the first image.

In step b1) second exposure metering area settings 136, 236, 336 are received by the processor of the mobile device 128, 228, 328 to cover a second sub-scene (126a, 226a, 326a) of a second scene (126, 226, 326) in the field of view of the camera or the entire second scene (126, 226, 326) in the field of view of the camera. The second exposure metering area may particularly be set by an app (due to the app's program code executed by the processor of the mobile device) downloaded to the mobile device or by a user of the mobile device.

In step b2) a second image of the second scene is captured. The capturing of the second image may be initiated by user action or may automatically be initiated (after the second exposure metering area settings were received), e.g. once a target object such as a color reference card 110, 220 is detected in the field of view of the camera based on position markers 112, 212 of the color reference card. The image acquisition may be supported by the processor of the mobile device 128, 228, 328 and the storing of the image may take place in a data storage device of the mobile device. For capturing the image, second auto-exposure settings may be used. The second auto-exposure settings may be determined based on the received second exposure metering area settings of step b1) or based on exposure metering area settings different therefrom. The mobile device may let a user or an app set a second exposure metering area (or rather pretend to the user or the app that a second exposure metering area may be set to be used for determining second auto-exposure settings only based thereon and not based on other parts of the second scene in the field of view), i.e. certain second exposure metering area settings are received in step b1), but actually used second auto-exposure settings when capturing the second image are nevertheless influenced by parts of the scene outside of the second sub-scene or second scene to which the second exposure metering area was set by the user or the app. In this case, the actually used second exposure metering area for determining second auto-exposure settings of the device thus is different from the second exposure metering area set by the user or app.

In step b3) a second intensity parameter is determined from the second image. The second intensity parameter may, e.g., be determined as the maximum, minimum, median or averaged intensity value of those pixels of an image corresponding to the second sub-scene or corresponding to a target object in the second image or of all pixels of the second image.

In step c) a suitability parameter is determined by using the first intensity parameter and the second intensity parameter,
wherein the suitability parameter being outside a predefined suitability range indicates non-suitability of the auto-exposure settings of the mobile device for performing a color based measurement and initiates a non-suitability action; and/or
wherein the suitability parameter being inside a predefined suitability range indicates suitability of the auto-exposure setting of the mobile device for performing a color based measurement and initiates a suitability action.

In case of the suitability parameter being outside a predefined suitability range, the mobile device may, e.g., be blocked from performing a color based measurement or, in case of under-or over-exposed image area(s) corresponding to the target object during the performance of a color-based measurement a user warning notification may be provided which may comprise a prompt to choose a lighter or darker background for placing the target object thereon than the one chosen for said image with under-and/or overexposed image area(s) when taking another image.

### List of reference numbers

110, 210 color reference card
112, 212 position marker
124, 224, 324 first scene
124a, 224a, 324a first sub-scene
124b, 224b, 324b first background scene
126, 226, 326 second scene
126a, 226a, 326a second sub-scene
126b, 226b, 326b second background scene
128, 228, 328 mobile device
134, 234, 334 first exposure metering area settings
136, 236, 336 second exposure metering area settings
140 evaluating the suitability of auto-exposure settings of a mobile device for performing a color based measurement
142 receiving first exposure metering area settings
144 capturing a first image of the first scene
146 determining a first intensity parameter from the first image
148 receiving second exposure metering area settings
150 capturing a second image of the second scene
152 determining a second intensity parameter from the second image
154 determining a suitability parameter

While this invention has been described as having an exemplary design, the present invention may be further modified within the spirit and scope of this disclosure. This application is therefore intended to cover any variations, uses, or adaptations of the invention using its general principles.

What is claimed is:

1. A method for evaluating the suitability of auto-exposure settings of a mobile device for performing a color based measurement, the mobile device having at least one camera for capturing an image representative of a scene; the method comprising:
a1) receiving first exposure metering area settings for a first sub-scene of a first scene in the field of view of the camera;
a2) capturing a first image of the first scene;
a3) determining a first intensity parameter from the first image;
b1) receiving second exposure metering area settings for a second sub-scene of a second scene or the entire second scene in the field of view of the camera;
b2) capturing a second image of the second scene;
b3) determining a second intensity parameter from the second image;
c) determining a suitability parameter as a function of the first intensity parameter and the second intensity parameter;
wherein (i) the suitability parameter being outside a predefined suitability range indicates non-suitability of the auto-exposure settings of the mobile device for performing a color based measurement and initiates a non-suitability action and/or (ii) the suitability parameter being inside the predefined suitability range indicates suitability of the auto-exposure setting of the mobile device for performing a color based measurement and initiates a suitability action; and
wherein either the second exposure metering area settings are different from the first exposure metering area settings or the second scene is different from the first scene.

2. The method according to claim 1, wherein the non-suitability action comprises providing a user warning notification and/or blocking a color based measurement with the mobile device and/or wherein the suitability action comprises unlocking a color based measurement with the mobile device.

3. The method according to claim 1, wherein the suitability parameter is the ratio of the first and second intensity parameter.

4. The method according to claim 1, wherein the first scene comprises the first sub-scene and a first background scene, and the second scene comprises the second sub-scene and a second background scene.

5. The method according to claim 4, wherein the first exposure metering area settings and the second exposure metering area settings are identical, the first sub-scene and the second sub-scene are identical and comprise a target object, and the first background scene and the second background scene are different.

6. The method according to claim 1, wherein the first scene and the second scene are identical while the first exposure metering area settings and the second exposure metering area settings are different in that the first exposure metering area settings are for the first sub-scene comprising a target object and the second exposure metering area settings are for the entire second scene.

7. The method according to claim 6, wherein the first intensity parameter is determined as the intensity of the first image and the second intensity parameter is determined as the intensity of the second image.

8. The method according to claim 1, wherein the first intensity parameter is determined as a function of a partial first image corresponding to a target object in the first sub-scene and the second intensity parameter is determined as a function of a partial second image corresponding to the target object in the second sub-scene.

9. The method according to claim 1, wherein the first scene and the second scene are identical and the first exposure metering area settings and the second exposure metering area settings differ from each other to cover different sub-scenes.

10. The method according to claim 6, wherein the first intensity parameter is determined as the intensity of the first image and the second intensity parameter is determined as the intensity of the second image.

11. The method according to claim 1, further comprising:
a4) determining a third intensity parameter from the first image;
b4) determining a fourth intensity parameter from the first or second image;
determining an admissibility parameter by using the third intensity parameter and the fourth image intensity parameter; and
wherein the suitability action is only initiated if the admissibility parameter is inside a predefined admissibility range.

12. The method according to claim 11, wherein the admissibility parameter is based on the ratio of the third and fourth intensity parameter.

13. The method according to claim 12, wherein:
the first scene comprises the first sub-scene and a first background scene, and the second scene comprises the second sub-scene and a second background scene;
the third image intensity parameter is determined as the intensity of the first background scene; and
the fourth image intensity parameter is determined as the intensity of the second background scene.

14. The method according to claim 12, wherein:
the first scene comprises the first sub-scene and a first background scene;
the third image intensity parameter is determined as the intensity of the first background scene; and
the fourth image intensity parameter is determined as the intensity of the first sub-scene.

15. The method according to claim 12, wherein:
the third image intensity parameter is determined as the intensity of the first sub-scene; and
the fourth image intensity parameter is determined as the intensity of the second sub-scene.

16. The method according to claim 1, further comprising:
a4) determining a third intensity parameter from the first image;
determining an admissibility parameter by using the third intensity parameter and the first intensity parameter; and
wherein the suitability action is only initiated if the admissibility parameter is inside a predefined admissibility range.

17. The method according to claim 1, wherein each of the first sub-scene and the second sub-scene comprises a target object wherein the target object is at least part of a color reference card.

18. The method according to claim 1, wherein the non-suitability action comprises providing a user warning notification with the mobile device.

19. The method according to claim 1, wherein the non-suitability action comprises blocking a color based measurement with the mobile device.

20. The method according to claim 1, wherein the suitability action comprises unlocking a color based measurement with the mobile device.

21. A mobile device having at least one camera for capturing an image representative of a scene, the mobile device being configured for performing the method according to claim 1.

22. A non-transitory computer-readable storage medium comprising instructions which, when the instructions are executed by a mobile device having a processor and a camera cause the processor of the mobile device to perform the method of claim 1.
